(19) United States
(12) Patent Application Publication
(10) Pub. No.: US 2015/0175961 A1
(43) Pub. Date: Jun. 25, 2015

(54) SYSTEM AND METHOD TO FACILITATE THE GROWTH OF STEM CELLS

(71) Applicants: Rasul Chaudhry, Rochester, MI (US); Ferman Chavez, Rochester, MI (US)

(72) Inventors: Rasul Chaudhry, Rochester, MI (US); Ferman Chavez, Rochester, MI (US)

(73) Assignee: OAKLAND UNIVERSITY, Rochester, MI (US)

(21) Appl. No.: 14/577,032

(22) Filed: Dec. 19, 2014

Related U.S. Application Data
(60) Provisional application No. 61/918,381, filed on Dec. 19, 2013.

(51) Int. Cl. C12N 5/0735; C12N 5/00

(52) U.S. Cl. CPC C12N 5/0606; C12N 5/0012; C12N 5/0062; C12N 2513/00; C12N 2500/50; C12N 2501/10; C12N 2501/20; C12N 2500/44

(57) ABSTRACT

A method and system to maintain adult or embryonic stem cells employing 3-D culture conditions is disclosed. Hydrogel scaffolds, composed of hydrophilic polymer networks are provided to emulate a fully hydrated native extracellular matrix and natural soft tissue. The hydrogel scaffolds constructs can be prepared to incorporate drugs, cytokines, and growth factors that promote proliferation, directed differentiation, and integration of cells to regenerate target tissue.

### CROSS-REFERENCE TO RELATED APPLICATIONS

This application claims the benefit of U.S. Provisional Application No. 61/918,381, filed on Dec. 19, 2013. The entire disclosure of the above application is incorporated herein by reference.

### FIELD

The present disclosure relates to system and method to facilitate the growth of stem cells and more particularly to a system and method to facilitate the growth of stem cells using a polymer scaffold system.

### BACKGROUND

This section provides background information related to the present disclosure which is not necessarily prior art.

The field of stem cell research emphasizes the importance of embryonic stem cells, since their pluripotent state allows for a wide array of translational and clinical applications. Research performed using animal models and more recently human clinical trials have shown promising potential of embryonic stem cells in cell therapies, repair of damaged tissues and organs, and in vitro disease modeling. However, these applications require routine and efficient expansion of pluripotent embryonic stem cells and controlled differentiation to obtain a homogenous population of cells. Recent reports have stressed the importance of extrinsic factors in stem cell fate determination, including interaction with tissue-specific microenvironments, the organization and composition of the extracellular matrix, cell-cell signaling, and the temporal and spatial gradient of soluble factors. This complex relationship between stem cell fate and their native microenvironment results in a large discrepancy between in vivo and in vitro culture conditions affecting the quality of cultured cells.

Conventionally, human and animal embryonic stem cells are grown in two-dimensional (2-D) plastic culture plates, coated with mouse embryonic fibroblast feeder layer or extracellular matrix components (such as gelatin and matrigel). Mouse embryonic stem cells can be maintained in their self-renewal state by the addition of soluble cytokines, such as leukemia inhibitory factor, to the culture media. Reliance on mouse embryonic fibroblasts, cytokines, or growth factors complicates the maintenance of stem cell lines due to the potential transmission of xenogeneic pathogens and the fluctuation of lot-to-lot quality. For this reason, the maintenance of the self-renewing state of pluripotent embryonic stem cells and induced pluripotent stem cells, remains a challenge. In addition to strict culture media and growth conditions, embryonic stem cells require regular passaging every two to three days, otherwise they are lost due to differentiation. Consequently, culturing embryonic stem cells is expensive and requires a high level of expertise. This is because 2-D culture conditions are not sufficient to mimic the intricacy of in vivo microenvironments. Specifically, the distribution of soluble factors in 2-D culture lacks the spatial gradient found in three-dimensional (3-D) microenvironments, which can alter cell fate determination. Furthermore, studies have shown that the extracellular matrix composition and organization can send mechanical signals for cell differentiation; cells plated in 2-D culture exhibit a changed morphology, which is sufficient to signal differentiation into a specific cell type.

Culturing of embryonic stem cells (embryonic stem cells) is laborious, technically challenging, and often leads to the loss of cell lines due to contamination and differentiation.

### SUMMARY

This section provides a general summary of the disclosure, and is not a comprehensive disclosure of its full scope or all of its features.

According to the present teachings, a method to maintain stem cell pluripotency employing 3-D culture conditions is disclosed. Hydrogel scaffolds, composed of hydrophilic polymer networks are provided to emulate a fully hydrated native extracellular matrix and natural soft tissue. The hydrogel scaffolds constructs can be prepared to incorporate drugs, cytokines, and growth factors that promote proliferation, directed differentiation, and integration of cells to regenerate target tissue. According to another teaching, a hydrogel scaffold for supporting stem cells is formed of polyethylene glycol. The polyethylene glycol is a building block for hydrogel synthesis when functionalized with the addition of crosslinking groups.

According to another teaching, a method of forming a hydrogel scaffold for supporting cells or stem cells is disclosed. The method includes chemically crosslinking hydrogels formed by a Michael-type addition when thiol-functionalized Dex (Dex-SH) is combined with an acrylate polymer, for example PEG functionalized with tetra-acrylate (PEG-4-Acr). Scaffold integrity can be altered by varying the degree of thiol substitution of Dex-SH, and molecular weight of the polymers.

According to the present teachings, a method of growing cells in a 3-D scaffolds for an extended period of time is presented. Embryonic stem cells morphology, pluripotency and upregulated embryonic stem cell specific markers are maintained.

According to present teachings, a method of providing three-dimensional (3-D) culture conditions mimicking an in vivo environment is taught. The method allows for the growth and maintenance of embryonic stem cell pluripotency without passaging. The method uses self-assembling scaffolds made of thiol-functionalized dextran (Dex-SH) and poly (ethylene glycol) tetra-acrylate (PEG-4-Acr) pluripotent growth of embryonic stem cells without manipulation for a prolonged period of time. The 3-D grown embryonic stem cells exhibited typical morphological and biochemical characteristics as well as expressed selected pluripotent genes at higher levels compared to the cells grown in traditional two-dimensional cultures. The method includes the differentiation into neural, myogenic, and osteogenic cell lineages of the 3-D grown embryonic stem cells.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1a-e represent cell viability and growth of embryonic stem cells encapsulated in self-assembling scaffolds;

FIGS. 2a-2c depict the effect of cell concentration on the rate of scaffold 10 degradation and embryonic stem cell colony size;

FIGS. 3a-3g represent the differentiation of 3-D scaffold grown embryonic stem cells into neural, myogenic, and osteogenic lineages;

FIG. 4 represents the expression of pluripotent markers of embryonic stem cells grown in 3-D scaffolds; and

FIG. 5 represents a system for transporting embryonic stem cells.

Corresponding reference numerals indicate corresponding parts throughout the several views of the drawings.

### DETAILED DESCRIPTION

Example embodiments will now be described more fully with reference to the accompanying drawings.

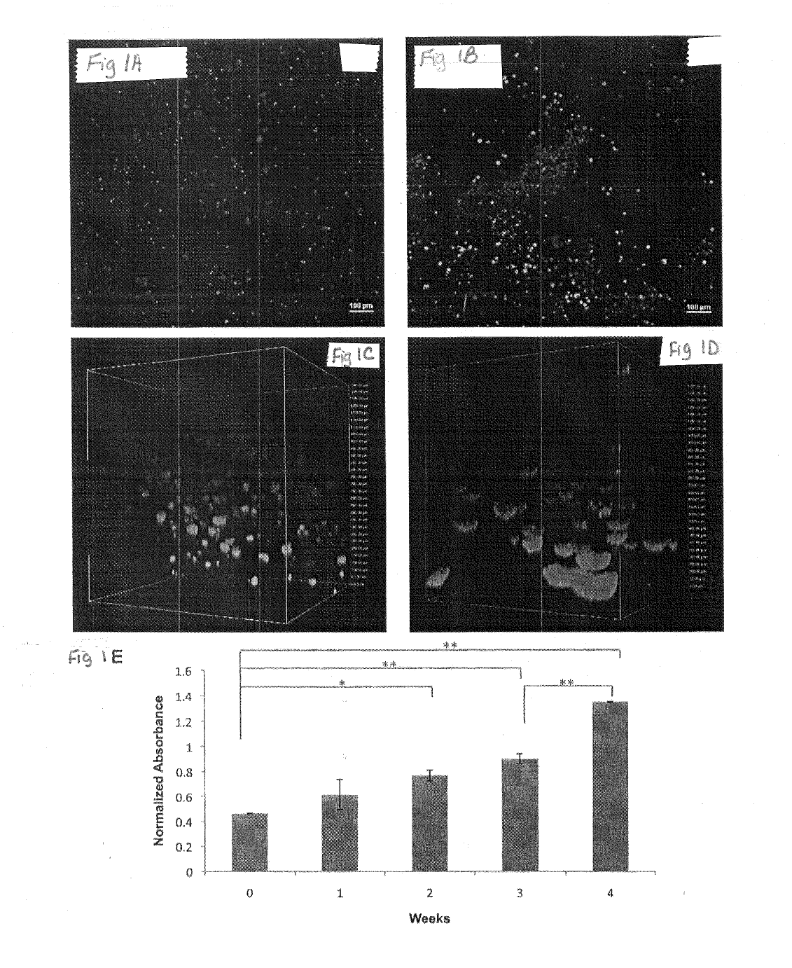

FIGS. 1a-e represent cell viability and growth of embryonic stem cells encapsulated in self-assembling scaffolds 10 according to the present teachings. The scaffolds 10, prepared with Dex-SH with between 2.5% and 10%, and preferably about 7.5% thiol substitution, PEG-4-Acr, and 2×10⁶ cells/ml, which can be fixed or float in media, were incubated in embryonic stem cell medium 14 for the cell growth. Periodically the scaffolds 10 were stained with propidium iodide for determination of viable and dead cells 12, and visualized by confocal microscopy. Shown are the fixed scaffolds 10 incubated for one day and four weeks, (A and B, respectively). Growth of embryonic or adult stem cells 12 in floating scaffolds 10 after one day and three weeks of incubation in embryonic stem cell medium 14 (C and D, respectively).

FIG. 1e represents the quantitative determination of stem cell 12 proliferation. The floating scaffolds 10 were stained by PB following zero, one, two, three, and four weeks of incubation and analyzed using a microplate reader. Data shown as the normalized absorbance±SEM, with a significant increase (*p<0.05 and **p<0.01 via ANOVA analysis) in cell number, compared to initial embryonic stem cell 12 viability at time 0.

As can be seen, the viability of 3-D grown embryonic stem cells was not affected by encapsulation or the process of scaffold 10 self-assembly. Cell growth increased over a period of three weeks, as evidenced by the increase in viable EYFP/GFP expressing cells in fixed scaffolds 10 (FIGS. 1a, b). Propidium iodide staining of the scaffolds one day after culturing showed a majority of viable cells with a small fraction (5%) of dead cells. Presence of dead cells could be due to encapsulation stress or damage caused by trypsinization of inoculated embryonic stem cells. When trypsinized stem cells 12 were tested by trypan blue staining, a similar fraction of dead cells (5-20%) was observed, indicating that encapsulation did not cause cell death. Unlike 2-D cell cultures, the encapsulated dead cells did notlyse during 3-D scaffold 10 incubation. In addition, the colony size of viable cells was restricted due to limited swelling of fixed scaffolds 10. If the scaffolds were allowed to swell freely, embryonic stem cell 12 growth and colony size can be increased. As seen, when embryonic stem cells 12 were cultured in the floating scaffolds, cell growth and colony size increased in a time dependent manner (FIGS. 1c, d).

Growth of encapsulated cells in the floating scaffolds was further confirmed by PrestoBlue staining and quantitative results are presented in FIG. 1e. Scaffold 10 grown embryonic or adult stem cells 12 showed a continuous increase in cell growth for four weeks, with a significant increase in proliferation shown at two, three and four weeks of incubation. Furthermore, the rate of cell proliferation significantly increased from three to four weeks, which could be attributed to the degradation of the scaffolds during this period. Cell growth and colony size were restricted during the early stages (up to three weeks) of incubation due to scaffold 10 integrity. Scaffolds that retain integrity longer than four weeks continue to support the undifferentiated growth of embryonic stem cells.

The polymer concentration and MW of Dex-SH and PEG-4-Acr, as well as the degree of thiol substitution of Dex-SH influenced the polymerization and degradation rate of scaffolds (data not shown). Optionally, Dex-SH (25 kDa) with 7.5% or 33% thiol substitution and PEG-4-Acr (20 kDa) at 5% w/v can be used as a scaffold.

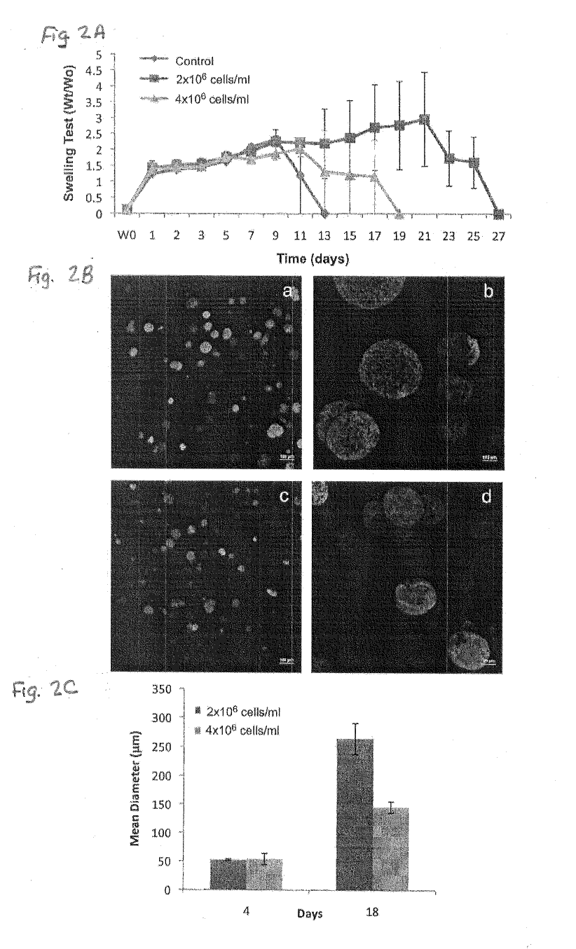

Scaffolds 10 prepared (with 7.5% thiol substitution of Dex-SH) with various cell concentrations, from 1×10⁶ cells/ml to 10×10⁶ cells/ml, exhibited different swelling profiles. The initial swelling capacity of the scaffolds was similar. The results of with scaffolds 10 containing 0, 2×10⁶ cells/ml and 4×10⁶ cells/ml shown in FIG. 2a indicated that swelling ratios were comparable until day nine. Control scaffolds without cells, degraded rapidly followed by scaffolds prepared with 4×10⁶ cells/ml and finally scaffolds with 2×10⁶ cells/ml. Scaffolds with the higher number of cells (4×10⁶ cells/ml) swelled to 2-fold of their initial weight and started to degrade on day nineteen of incubation. Whereas, scaffolds with lower number of cells (2×10⁶ cells/ml) swelled to nearly three times of their initial weight and started to degrade on day twenty-seven of incubation. Furthermore, the cells grew rapidly with larger colony size in scaffolds with lower cell concentration (2×10⁶ cells/ml) (FIGS. 2b; a, b) as compared to the higher cell concentration (4×10⁶ cells/ml) (FIGS. 2b; c, d). Quantitative analysis of colony size showed a two-fold increase in the mean diameter of colonies in scaffolds prepared with lower number of cells (FIG. 2c).

FIGS. 2a and 2b represent the effect of cell concentration on the rate of scaffold 10 degradation and stem cell 12 colony size. As shown in FIG. 2a, floating scaffolds 10 were prepared using Dex-SH with 7.5% thiol substitution, PEG-4-Acr, and embryonic stem cells (0, 2×10⁶ or 4×10⁶ cells/ml). The scaffolds 10 were incubated in embryonic stem cell medium 14 and compared for degradation using swelling tests on every other day. Data expressed as the mean swelling ratio±SEM of a triplicate experiment.

As shown in FIG. 2b, the determination of embryonic stem cell 12 colony 16 size, for scaffolds incubated in embryonic stem cell medium 14 can be visualized by confocal microscopy. Shown are the scaffolds 10 with 2×10⁶ cells/ml (a and b) and 4×10⁶ cells/ml (c and d), which were incubated in the medium 14 for four and eighteen days, respectively. (C) The colony size was quantified and expressed as mean diameter (μm)±SEM of a triplicate experiment.

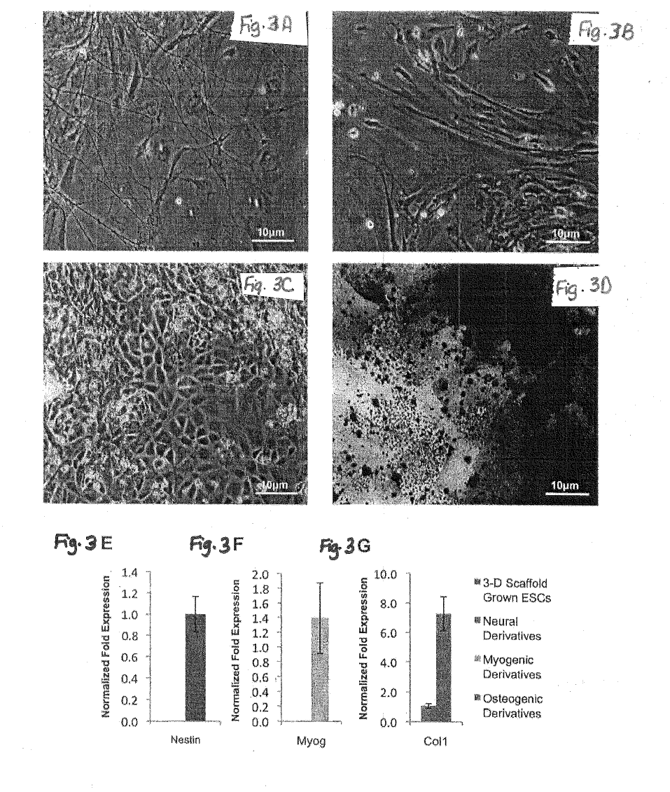

FIGS. 3a-3e represent the differentiation of 3-D scaffold 10 grown embryonic stem cells 12 into neural, myogenic, and osteogenic lineages. (A-C) After culturing in scaffolds 10 for three weeks, embryonic stem cells 12 were passaged by sub-culturing on mouse embryonic fibroblasts. After five passages, they were used to prepare Embryoid bodies and differentiated into various cell lineages using selective culture media. Morphology of embryonic stem cell 12 derivatives is shown for neural, myogenic, and osteogenic cells (A, B, and C, respectively). (D) Deposition of calcium in osteogenic embryonic stem cell 12 derivatives. Osteogenic cells were cultured for four weeks and subjected to von Kossa staining, visualized by light microscopy (10X). Osteogenic cell-specific extracellular matrix stained dark brown. (E-G) Expression of cell-specific markers in embryonic stem cells derivatives (shown in A-D). Embryonic stem cell 12 derivatives expressed Nestin, Myog, and Col1 markers for neural, myogenic, and osteogenic cells (E, F, and G, respectively) were distinguishable after growth in the 3-D scaffold 10. Results represent the normalized fold expression±SEM of triplicate experiments.

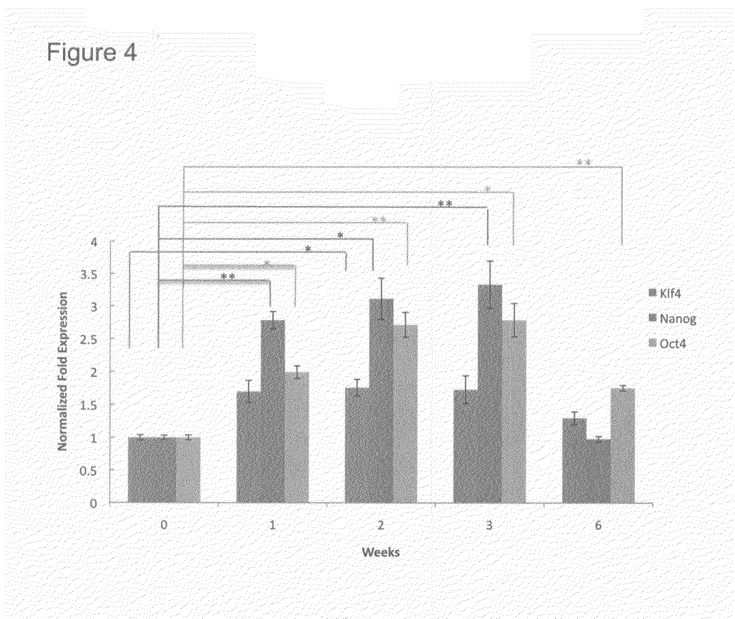

FIG. 4 represents expression of pluripotent markers of embryonic stem cells 12 grown in 3-D scaffolds 10. Cells cultured in scaffolds 10 prepared with Dex-SH with 33% thiol substitution, were analyzed for expression of selected markers, Oct 4, Nanog, and Klf4, by qRT-PCR after zero, one, two, three, and six weeks of embryonic stem cell 12 growth. Results of triplicate experiments were expressed as the normalized fold expression±SEM, where a significant (*p<0.05 and **p<0.01) increase of pluripotent marker expression was compared to initial 2-D grown embryonic stem cells (day zero), via ANOVA analysis. Results depicted showed upregulation of Oct4 and Nanog, while Klf4 was maintained at the same level until the onset of degradation, at which point the expression of pluripotent markers decreased to initial levels.

Pluripotency potential of 3-D scaffold 10 cultured embryonic stem cells was determined by differentiation via the embryoid body. Embryonic stem cells 12 grown in 3-D scaffolds 10 differentiated into neural, myogenic, and osteogenic cell types akin to embryonic stem cells grown in 2-D cultures. The results of differentiation depicted in FIG. 3 indicate that neural derivatives of embryonic stem cells 12 displayed neurofilaments (FIG. 3a), while myogenic derivatives had spindle shaped morphology (FIG. 3b). Osteogenic derivatives exhibited the cobblestone appearance of osteoblast cells and were positive for calcium deposition as determined by von Kossa staining (FIGS. 3c, d). Furthermore, quantitative real-time polymerase chain reaction (qRT-PCR) analyses revealed expression of cell-specific differentiation markers such as Nestin, Myogenin (Myog), and Collagen type 1 (Col1), for neural, myogenic, and osteogenic derivatives of 3-D grown embryonic stem cells (FIGS. 3e-g, respectively). The expression of these cell-specific markers signified that 3-D grown embryonic stem cells maintained differentiation potential similar to embryonic stem cells grown in 2-D cultures.

To further confirm whether 3-D scaffold 10 (prepared using 33% thiol substitution) grown embryonic stem cells maintained pluripotency, expression of selected embryonic stem cell-specific markers was analyzed using qRT-PCR. The results shown in FIG. 4 indicated that expression of Oct4 and Nanog was two to three fold higher, in 3-D grown embryonic stem cells as compared to cells grown under 2-D culture conditions, until the onset of degradation that occurred at about 6 weeks. The expression of these markers significantly and successively increased in 3-D grown embryonic stem cells during one, two and three weeks. Whereas, the expression of Klf4 in 3-D grown embryonic stem cells remained slightly above the level of 2-D grown embryonic stem cells throughout incubation, with a significant increase at two weeks. At six weeks, with the concomitant start of degradation of the scaffolds, expression of pluripotency markers gradually decreased to the levels of 2-D grown embryonic stem cells.

The rate of 3-D hydrogel scaffold 10 formation was dependent upon several factors. The results showed that the greater the degree of thiol substitution, the faster the polymerization of Dex-SH and PEG-4-Acr yielded a 3-D scaffold. Furthermore, the ratio and amount of Dex-SH and PEG-4-Acr used also affected the formation and swelling properties of the hydrogel scaffolds. Dex-SH and PEG-4-Acr at 5% w/v Dec-SH and PET-4-Acr polymer concentrations viable to provide a scaffold 10 microenvironment with a greater degree of flexibility and suitability for embryonic stem cells growth.

Optimal conditions for scaffold 10 polymerization, cell encapsulation, and cell viability were achieved by varying polymer concentration, percentage of thiol substitution, and cell concentration. Under these conditions, cell mixing with one of the components, PEG-4-Acr, prior to scaffold 10 molding yielded homogeneous distribution of cells. These conditions also promoted embryonic stem cell 12 growth in both fixed and floating scaffolds, and did not interfere with nutrient diffusion generally associated with seeding pre-formed scaffolds 10. The 3-D scaffold 10 grown embryonic stem cells 12 displayed undifferentiated compact round colony morphology, even during prolonged periods of culture. Self-assembly of the Dex-SH and PEG-4-Acr scaffold 10 did not require a mutagenic catalyst and embryonic stem cells were maintained for over six weeks, subject to the integrity of the scaffold. The-self-assembling scaffolds supported embryonic stem cell 12 growth at concentrations even lower than 1×10⁶/ml.

Embryonic stem cell 12 self-renewal was correlated with the integrity of the scaffolds as the cells were differentiated upon complete degradation of the scaffolds. Scaffold 10 integrity also influenced the cell proliferation rate and colony size. Embryonic stem cells grew at a slower rate with smaller colony size in fixed scaffolds until the scaffold 10 started to degrade. However, cell growth rate and colony size was less restricted in floating scaffolds.

In addition to the concentration of the scaffold 10 components and degree of thiol substitution, cell concentration significantly affected the rate of scaffold 10 degradation. Scaffolds 10 without cells 12 degraded the earliest, around day 13, suggesting that the addition of cells improved the stability of the scaffolds. Moreover, scaffolds 10 prepared with a higher concentration of embryonic stem cells 12 degraded faster and swelled less than scaffolds 10 with lower concentration of embryonic stem cells 12. Correspondingly, embryonic stem cell 12 colony size was smaller in scaffolds with higher concentration of cells as compared to the scaffolds prepared with the lower cell concentration. Furthermore, encapsulated embryonic stem cells in the floating scaffolds grew rapidly with larger colony sizes as compared to the fixed scaffolds of similar composition. These results suggested that the swelling plasticity of the self-assembling scaffolds favorably promoted the growth and maintenance of embryonic stem cells 12.

Cells 12 grown in 3-D scaffolds 10 for a period of six weeks not only exhibited typical characteristics of embryonic stem cells 10, but also differentiated into specific cell lineages either directly or upon passaging on mouse embryonic fibroblasts in 2-D culture via Embryoid bodies directed differentiation. Differentiated derivatives of embryonic stem cells 10 exhibited lineage specific morphological, biochemical, and molecular properties. Embryonic stem cells 12 cultured in the 3-D scaffolds 10 differentiated into neural, myogenic, and osteogenic cell types and expressed Nestin, Myog, and Col1, respectively. These results indicated that the self-assembling scaffolds 10 supported the self-renewal and pluripotency of embryonic stem cells, even after prolonged periods of culturing.

3-D scaffold 10 grown embryonic stem cells would display typical expression of pluripotent markers. Surprisingly, the expression of selected pluripotency markers such as Oct4 and Nanog was higher in 3-D scaffold 10 cultured embryonic stem cells as compared to 2-D grown cells. In this regard, expression remained at higher levels throughout the extended growth period, up to six weeks, until the onset of scaffold 10 degradation.

The period of undifferentiated growth of embryonic stem cells in the 3-D scaffolds could be controlled by altering scaffold 10 degradability, which was dependent on the degree of thiol substitution of Dex-SH and concentration and MW of the scaffold 10 components as well as the number of cells used.

Presented according to the present teachings is a robust system for 3-D culturing of embryonic stem cells for extended periods without passaging or manipulation. These improvements in the maintenance of embryonic stem cells will help to promote their increased use for translational research, disease modeling, stem cell 12 therapies, and regenerative medicine.

The mouse embryonic stem cell 12 line 7AC5, labeled with enhanced yellow fluorescent protein (EYFP), (ATCC; Manassas, Va.) was cultured on irradiated mouse embryonic fibroblast feeder layer with embryonic stem cell medium 14 containing leukemia inhibitory factor.

The self-assembling scaffold can be prepared by Dex (25 kDa, M_{W}/M_{N}˜1.30, Sigma, St. Louis, Mo.) was functionalized with pendant SH groups at differing degrees of thiol substitution ranging from 4% to 34%. The percentage of thiol substitution can be controlled by varying the amount of 4-nitrophenylchloroformate in the reaction. Dex-SH can be characterized by ¹HNMR spectroscopy using a 400 MHz Bruker Avance II spectrometer.

Hydrogel scaffolds can be formed by mixing Dex-SH with PEG-4-Acr (20 kDa, Creative PEGWorks Winston Salem, N.C.) via a Michael addition reaction. Scaffolds 10 can be prepared with final polymer concentrations off 5% w/v. The molar ratio of thiol to acrylate groups used can be 1:1.

To encapsulate cells 12, Dex-SH and PEG-4-Acr can be dissolved separately in culture medium 14 and mixed with embryonic stem cells 12 (70% confluency), at a concentration of 2×10⁶ cells/ml or 4×10⁶ cells/ml. The resulting mixture can be transferred to either a well of a 96-well plate or 1 cc syringe for polymerization to produce fixed or floating scaffolds, respectively. The encapsulated embryonic stem cells can be cultured in embryonic stem cell medium. The medium 14 optionally can be changed daily or as needed. The 2-D and 3-D cell growth was monitored by phase-contrast and confocal microscopy and analyzed by NIS Elements AR software.

The degradation rate of the scaffolds can be determined by swelling tests. For example, floating scaffolds can be prepared by encapsulating embryonic stem cells at concentrations of 0, 2×10⁶, or 4×10⁶ cells/ml. The initial weight (Wᵢ) of the scaffold 10 should be measured, before incubation in a 24-well plate with 1.5 ml embryonic stem cell medium. The scaffolds can then be removed from the medium 14 to record the swollen weight (Wₛ) every other day. The swelling ratio described above is defined as the difference between Wₛ and Wᵢ divided by Wᵢ.

Cell viability was determined qualitatively by propidium iodide (PI) (Fisher Scientific, Pittsburgh, Pa.) staining in triplicate experiments, and was visualized using confocal microscopy. The quantitative analysis of cell growth in the scaffolds was determined by PrestoBlue assays (Invitrogen, Carlsbad, Calif.), following the manufacturer's instructions. The scaffolds were incubated in PrestoBlue solution for four hours, before measuring the absorption of the solution at 570 nm and 600 nm wavelengths with the Epoch microplate reader (BioTek, Winooski, Vt.). PrestoBlue, a resazurin-based solution, was reduced proportional to the number of metabolically active cells to fluorescent resorufin.

Embryoid bodies were prepared from embryonic stem cells 12 by the hanging drop method. For differentiation into a myogenic phenotype, Embryoid bodies were cultured in embryonic stem cell growth medium. For neural and osteogenic differentiation, Embryoid bodies were treated with 10⁻⁷ M trans-retinoic acid (RA) and cultured in embryonic stem cell medium 14 supplemented with B-27 (10 μl/ml; Invitrogen, Carlsbad, Calif.), L-glutamine (10 μl/ml; Sigma, St. Louis, Mo.) and penicillin/streptomycin (1 μl/ml; Sigma, St. Louis, Mo.) and β-glycerol phosphate (10 μl/ml; Sigma, St. Louis, Mo.), and ascorbic acid (10 μl/ml; Sigma, St. Louis, Mo.), respectively. Cell morphology was monitored by light microscopy on a daily basis. Osteogenic cells were also analyzed for calcium deposition by von Kossa staining.

Gene expression studies can be performed using qRT-PCR. RNA was isolated from cells using the RNeasy Mini kit (Qiagen, Germantown, Md.). Encapsulated cells 10 were flash frozen with liquid nitrogen, grinded into a fine powder using a mortar and pestle, and homogenized using the QIAshredder column. RNA was purified by treating with RNase-free DNase (Promega, Madison, Wis.) and cDNA was synthesized with the iScript kit (BioRad, Hercules, Calif.). PCR reactions were performed in a 10 μl reaction volume on the using the BioRad CFX90 Real-Time PCR system and SsoAdvanced SYBR Green Supermix. The specific PCR conditions used were as follows: polymerase activation three minutes at 95° C., forty cycles of denaturation, fifteen seconds at 95° C.; annealing, twenty seconds at 60° C.; and melt curve, five seconds/step at 60-95° C. The markers used in this study represent pluripotency as well as neural, myogenic, and osteogenic lineages. Primers (IDT Technologies, Coralville, Iowa) are listed in the supplemental material (Table S1). All reactions were prepared in triplicate and normalized to reference gene expression of Gapdh and β-Actin.

All quantitative data were expressed as mean±SEM. One-way ANOVA analysis was performed to compare the relative gene expression of pluripotency and differentiation markers in qRT-PCR studies. Results with a p-value less than 0.05 was considered to be significant (*p<0.05 and **p<0.01). All analyses were performed using SPSS version 11.5.

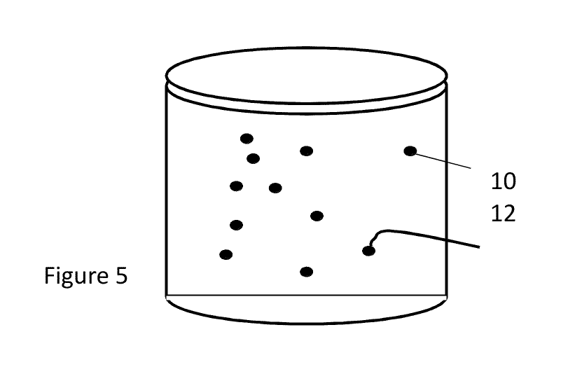

Shown in FIG. 5 is a closable container having a hydrogel scaffold within the closable container. The hydrogel scaffold can be polyethylene glycol and has a hydrophilic polymer networks. Disposed within the hydrophilic scaffold is the stem cell. The hydrophilic scaffold is configured to emulating a fully hydrated native extracellular matrix and natural soft tissue in the hydrogel scaffold. The hydrogel scaffold comprises at least one of drugs, cytokines, and growth factors that promote proliferation, directed differentiation, and integration of cells to regenerate target tissue. The hydrogel within the container is maintained during shipping at one of liquid nitrogen temperature (below −346 degrees F.), dry ice temperature (below 109.8 degrees F.), or ambient temperature during shipping.

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The disclosure of all patents and patent applications cited in this disclosure are incorporated by reference herein. The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method to maintain stem cell pluripotency employing 3-D culture conditions comprising:
forming a hydrogel scaffold, composed of hydrophilic polymer networks;
suspending the stem cell within the hydrogel scaffolds; and
emulating a fully hydrated native extracellular matrix and natural soft tissue in the hydrogel scaffold.

2. The method according to claim 1, wherein the hydrogel scaffold comprises at least one of drugs, cytokines, and growth factors that promote proliferation, directed differentiation, and integration of cells to regenerate target tissue.

3. The method according to claim 1, wherein the hydrogel scaffold comprises polyethylene glycol.

4. The method according to claim 3, wherein the polyethylene glycol is functionalized with the addition of crosslinking groups.

5. A method to maintain stem cell pluripotency employing 3-D culture conditions comprising:
chemically crosslinking hydrogels formed by a Michael-type addition when thiol-functionalized Dex (Dex-SH) combined with an acrylate polymer, PEG functionalized with tetra-acrylate (PEG-4-Acr), wherein the hydrogel scaffold comprises at least one of drugs, cytokines, and growth factors that promote proliferation, directed differentiation, and integration of cells to regenerate target tissue;
and
disposing the stem cell within the hydrogel.

6. The method according to claim 5, further comprising altering the hydrogel integrity by altering by the degree of thiol substitution of Dex-SH, and molecular weight of the polymers.

7. The method according to claim 5, wherein the hydrogel comprises self-assembling scaffolds made of thiol-functionalized dextran (Dex-SH) and poly (ethylene glycol) tetra-acrylate (PEG-4-Acr); and
pluripotent growth of stem cells without manipulation for a predetermined period of time.

8. The method according to claim 7, further comprising the step of differentiating the cells into neural, myogenic, and osteogenic cell lineages of the 3-D grown stem cells.

9. A method to maintain stem cells pluripotency comprising:
forming a hydrogel scaffold, composed of hydrophilic polymer networks;
suspending the plurality of stem cells within the hydrogel scaffolds for greater than 2 weeks; and
emulating a fully hydrated native extracellular matrix and natural soft tissue in the hydrogel scaffold, wherein the stem cells express markers, Oct 4, Nanog, and Klf4, after two weeks of stem cell growth.

10. The method according to claim 9, wherein the hydrogel scaffold comprises at least one of drugs, cytokines, and growth factors that promote proliferation, directed differentiation, and integration of cells to regenerate target tissue.

11. The method according to claim 9, wherein the hydrogel scaffold comprises polyethylene glycol.

12. The method according to claim 11, wherein the polyethylene glycol is functionalized with crosslinked groups.

13. A method to maintain embryonic stem cell pluripotency employing 3-D culture conditions comprising:
chemically crosslinking hydrogels formed by a Michael-type addition of thiol-functionalized Dex (Dex-SH) combined with an acrylate polymer, PEG functionalized with tetra-acrylate (PEG-4-Acr); and
disposing the embryonic stem cell within the hydrogel.

14. The method according to claim 13, further comprising altering the hydrogel integrity by altering one of the degree of thiol substitution of Dex-SH and the molecular weight of the polymers.

15. The method according to claim 13, wherein the hydrogel comprises self-assembling scaffolds made of thiol-functionalized dextran (Dex-SH) and poly (ethylene glycol) tetra-acrylate (PEG-4-Acr); and further comprising pluripotent growth of stem cells without manipulation for a prolonged period of time.

16. The method according to claim 15, further comprising the step of differentiating the stem cells into neural, myogenic, and osteogenic cell lineages of the 3-D grown stem cells.

17. A method of shipping stem cells comprising:
providing a closable container
forming a hydrogel scaffold within the closable container, the hydrogel scaffold composed of a hydrophilic polymer networks;
suspending the stem cell within the hydrogel scaffolds;
emulating a fully hydrated native extracellular matrix and natural soft tissue in the hydrogel scaffold; and
shipping the closable container.

18. The method according to claim 17, wherein the hydrogel scaffold comprises at least one of drugs, cytokines, and growth factors that promote proliferation, directed differentiation, and integration of cells to regenerate target tissue.

19. The method according to claim 17, wherein the hydrogel is maintained at one of below −346° F., 109.8° F. or ambient temperature during shipping.

20. The method according to claim 19, wherein the hydrogel comprises crosslinked polyethylene glycol.
